(19) United States
(12) Patent Application Publication
(10) Pub. No.: US 2025/0121347 A1
(43) Pub. Date: Apr. 17, 2025

(54) WATER-SOLUBLE GINGER OLEORESIN MICROCAPSULES AND A METHOD OF PREPARING THE SAME AND APPLICATION THEREOF

(71) Applicant: HENAN ZHONGDA HENGYUAN BIOTECHNOLOGY STOCK CO., LTD., Luohe City (CN)

(72) Inventors: Honglong LI, Luohe City (CN); Mingming WANG, Luohe City (CN); Ziheng JIN, Luohe City (CN); Yanjun WEN, Luohe City (CN); Linzheng LI, Luohe City (CN); Chunfeng YU, Luohe City (CN); Xiaosong XU, Luohe City (CN); Di WANG, Luohe City (CN)

(21) Appl. No.: 18/913,948

(22) Filed: Oct. 11, 2024

(30) Foreign Application Priority Data
Oct. 13, 2023 (CN) 202311327005.0

(51) Int. Cl. B01J 13/04; A61K 8/11; A61K 9/50

(52) U.S. Cl. CPC B01J 13/043; A61K 8/11; A61K 9/5036

(57) ABSTRACT

The invention pertains to the technical field of natural product microencapsulation, specifically focusing on a water-soluble ginger oleoresin microcapsule, along with its preparation method and applications. This invention utilizes modified starch, plant polysaccharides, and similar materials as wall materials, with ginger oleoresin serving as the core material. The ginger oleoresin microcapsules are produced through processes including molecular coating, emulsification, high-pressure homogenization, constant temperature standing, and drying. The resulting product is characterized by a high gingerol content, excellent water solubility, low spiciness in solution, and strong stability in acidic environments. It fulfills the requirements of high stability, no precipitation, low spiciness, and a pleasant thermal sensation in low pH environments, making it suitable for use in food, beverages, health foods, medicines, cosmetics, and other applications.

This application claims priority to Chinese Patent Application No. 202311327005.0, filed on Oct. 13, 2023, which is incorporated by reference for all purposes as if fully set forth herein.

### TECHNICAL FIELD

The present invention relates to the technical field of natural product microencapsulation, specifically to water-soluble ginger oleoresin microcapsules, their preparation method, and applications.

### BACKGROUND TECHNIQUE

Ginger, a perennial herb of the Zingiberaceae family, is also known as Junjiang or Baijiang. China is recognized as both the origin and one of the major producers of ginger. Ginger has been cultivated in China for over 2,000 years and is widely distributed throughout the country. It is rich in nutrients, including gingerol, starch, dietary fiber, protein, fats, and trace elements. In daily life, ginger is primarily used as a seasoning, enhancing both aroma and flavor. Additionally, it is widely used in traditional Chinese medicine for the treatment and prevention of various ailments. “The Compendium of Materia Medica” notes that “ginger is hot in nature, spicy in taste, and enters the heart, kidney, spleen, stomach, and lung meridians.” It is believed to have effects such as warming the middle and dispelling cold, restoring yang and unblocking meridians, and reducing dampness and inflammation. “The Shennong's Herbal Classic” classifies ginger as a top-grade product, stating that “it can be used as a vegetable, condiment, fruit, and medicine, offering broad benefits.” Currently, ginger is widely utilized in the fields of food, medicine, health supplements, and cosmetics.

Gingerol is a collective term for the “spicy” compounds found in ginger, typically appearing as yellow to brown oily substances. It is insoluble in water but soluble in lipophilic solvents such as ethanol, benzene, and ether. Gingerol is a mixture of various compounds, including gingerols, shogaols, gingerones, gingeroleones, gingerdiols, gingerdiones, new gingerols, and flavonoids containing 3-methoxy-4-hydroxyphenyl groups. As the principal active component in ginger, gingerol exhibits significant antioxidant and anti-inflammatory properties. It also promotes stomach health, liver protection, bile secretion, and offers antiemetic, anti-ulcer, anticoagulant, and antitumor effects. Consequently, gingerol is widely used in clinical medicine and health foods.

Ginger oleoresin is a viscous, yellow-brown to brownish-yellow semi-fluid obtained by extracting and concentrating ginger. It has a strong ginger aroma, with main ingredients like fat-soluble substances such as ginger essential oil and gingerol, and is mostly used as condiments. It is also commonly used in health foods and medicines where antioxidant and anti-inflammatory properties are desired.

As the economy develops, consumer focus on health has increased, leading to a greater demand for diversified, functional foods and healthy raw materials sourced from nature. The need to use food-grade or highly safe natural raw materials to prepare ginger oleoresin microcapsule powder with high gingerol content, reduced spiciness, good water solubility, and stable application in low pH environments (such as beverages, oral liquids, candies, solid drinks, cold drinks, etc.) has become an urgent market demand with significant potential.

Relevant literature and patents discuss the preparation of ginger oleoresin microcapsule powder. For instance, the study titled “The Effect of Spray Drying Conditions on Ginger Oleoresin Microcapsules” (published in “Food Science”, 2009, Issue 3, by the School of Food Science and Engineering, Yangzhou University) details the preparation of ginger oleoresin microcapsule powder. The process involved using ginger as the raw material, extracting the ginger oleoresin with ethanol, and vacuum drying. β-cyclodextrin and Tween-80 were used as wall materials, and the microcapsule powder was prepared using emulsification, homogenization, and spray drying techniques. The study primarily focused on examining the effects of various spray drying conditions on the ginger oleoresin microcapsules. CN 101473954A discloses a ginger oleoresin microcapsule and its preparation method; CN 113424958A discloses a high gingerol content taste-masking microcapsule and its preparation method; CN 113952314A discloses a tea polyphenol-gingerol combination microparticle, along with its preparation method and application; CN 109511983A discloses a ginger oleoresin microcapsule and its preparation method; and CN 102895189A discloses a gingerol liposome and its preparation method, among others. However, although these processes improve the dissolution of ginger oleoresin to some extent, they are often complex and involve substantial use of chemically synthesized surfactants, such as Tween, monoglycerides, sucrose esters, and polyethylene glycol, raising food safety concerns. Additionally, many products continue to struggle with issues such as low gingerol content, strong spiciness, poor water solubility, and low stability in low pH environments. It cannot meet the health and safety demands of food products, nor the specific quality requirements of beverages, oral liquids, candies, solid drinks, cold drinks, and other foods for ginger oleoresin microcapsule powder in terms of high gingerol content, low spiciness, good water solubility, and stability in low pH environments.

### SUMMARY OF THE INVENTION

To overcome the deficiencies and limitations of the prior art, the primary purpose of the present invention is to provide a method for preparing water-soluble ginger oleoresin microcapsules that avoids the use of chemical surfactants and solvents. Instead, it employs widely accepted food-grade raw materials and auxiliary materials. Through techniques such as molecular coating, emulsification, homogenization, and microcapsule drying, this method produces a ginger oleoresin microcapsule product with high gingerol content, reduced spiciness, good thermal sensation, excellent water solubility, and stability in low pH environment.

Another purpose of the present invention is to provide a water-soluble ginger oleoresin microcapsule prepared by the aforementioned method. This microcapsule exhibits high gingerol content, low spiciness in aqueous solutions, good water solubility, high clarity in solution, and excellent stability in low pH environments, making it suitable for the specific requirements of beverages, oral liquids, candies, solid drinks, cold drinks, and other food and medicinal products.

Another purpose of the present invention is to provide the application methods for the aforementioned water-soluble ginger oleoresin microcapsules.

In order to achieve the above object, the present invention provides water-soluble ginger oleoresin microcapsules and a method of preparing the same, characterized by comprising the following steps:
(1) Mix cyclodextrin with water and heat until fully dissolved to obtain a cyclodextrin solution. Then, add ginger oleoresin under high shear conditions to emulsify and disperse, forming a ginger oleoresin-cyclodextrin dispersion;
(2) Mix the wall material with water to prepare a solution, then heat to obtain a wall material solution;
(3) Add the ginger oleoresin-cyclodextrin dispersion obtained in step (1) to the wall material solution from step (2) under high shear conditions to emulsify and disperse, adjusting the system pH to 6.5-10.0. Then, add flavonoids and continue emulsifying and dispersing to obtain a ginger oleoresin emulsion. Finally, high-pressure homogenize the ginger oleoresin emulsion to obtain a ginger oleoresin homogenate.
(4) Allow the ginger oleoresin homogenate obtained in step (3) to stand at a constant temperature, and then dry it to obtain water-soluble ginger oleoresin microcapsules.

The cyclodextrin described in step (1) is at least one of α-cyclodextrin, β-cyclodextrin, and γ-cyclodextrin; The mass ratio between ginger oleoresin and cyclodextrin in step (1) is (1-2): 1; The heating temperature in step (1) is 40-90° C.; The concentration of the cyclodextrin solution in step (1) is 10-50 wt. %; The temperature of the ginger oleoresin in step (1) is 20-80° C., preferably 40-70° C.; The speed of the emulsification dispersion in step (1) is 500 rpm to 20,000 rpm, preferably 3,000 rpm to 10,000 rpm; The emulsification dispersion time is 0.5-60 minutes, preferably 10-30 minutes, and the emulsification dispersion temperature is 40-90° C.

The wall material described in step (2) is at least one of gum arabic, pullulan, modified starch, gum dawa, peach gum, fucoidan, and chitosan; The modified starch is preferably at least one of sodium starch octenyl succinate and hydroxypropyl starch; The wall material solution in step (2) preferably include a filler; The filler can be at least one of maltodextrin, microcrystalline cellulose, glucose syrup, microporous starch, resistant dextrin, white sugar, lactitol, erythritol, maltitol, sorbitol, stachyose, beet water-soluble fiber, trehalose, oligomaltose, and lactose; The concentration of the wall material in the wall material solution is 5-70 wt. %, and the concentration of the filler is 5-70 wt. %; The temperature of the wall material solution in steps (2) and (3) is 40-90° C., preferably 40-80° C.

The mass ratio of the ginger oleoresin-cyclodextrin dispersion to the wall material solution in step (3) is 1:(2.5-3); The speed of emulsification dispersion in step (3) ranges from 500 rpm to 20,000 rpm, preferably from 3,000 rpm to 10,000 rpm; The time for emulsification dispersion is 0.5-60 minutes, preferably 10-30 minutes. The emulsification dispersion temperature is 40-90° C., preferably 40-80° C.; The reagent used for adjusting the pH of the system in step (3) is at least one of sodium carbonate, sodium bicarbonate, potassium carbonate, sodium hexametaphosphate, sodium tripolyphosphate, sodium polyphosphate, sodium pyrophosphate, and sodium citrate; The pH of the system in step (3) is preferably 7.0-9.0; The flavonoids used in step (3) include at least one of EGCG, hesperidin, dihydromyricetin, myricetin, rutin, and quercetin, with an additional amount of 0.01-1.5 wt. % relative to the final product. The pressure for high-pressure homogenization in step (3) ranges from 0.1-100 MPa, preferably from 20 MPa to 90 MPa; The number of high-pressure homogenizations in step (3) is 1 to 3 times, preferably 1 to 2 times.

The temperature for constant temperature standing in step (4) is 30-90° C., preferably 40-80° C.; The time for constant temperature standing in step (4) is 1 to 12 hours, preferably 4 to 8 hours; The drying method described in step (4) can be centrifugal spray drying, pressure spray drying, freeze drying, or hot air drying; The preferred drying method is centrifugal spray drying; The inlet air temperature for centrifugal spray drying is preferably 160-200° C., and the outlet air temperature is preferably 70-100° C.

The application of the water-soluble ginger oleoresin microcapsule in food, medicine, or cosmetics. Wherein, the present invention imposes no special restrictions on the form of the food; it can be added to liquid or solid food, with a preference for preparation as a beverage. If the food is a health product, the present invention imposes no special restrictions on its dosage form, which can be in the form of capsules, tablets, oral liquids, etc.

Principle of the Present Invention: Cyclodextrin molecules have a slightly conical, hollow, cylindrical three-dimensional ring structure. The upper end (wider opening) of the hollow structure is composed of secondary hydroxyl groups at C2 and C3, while the lower end (narrower opening) consists of primary hydroxyl groups at C6, making them hydrophilic. The hollow cavity forms a hydrophobic region due to the shielding effect of C—H bonds, exhibiting a certain degree of non-polarity. The hollow structure within the cyclodextrin molecule can encapsulate other molecules of similar molecular weight. The present invention utilizes cyclodextrin molecule pre-coating technology, leveraging the gingerol components in ginger oleoresin that are easily embedded into the cyclodextrin's hollow structure to pre-coat the ginger oleoresin. Typically, molecules embedded in the cyclodextrin's hollow structure are released very slowly, thereby reducing the spiciness to a certain extent.

Due to the solubility defect of cyclodextrin, which is poor water solubility, if the effective ingredients in ginger oleoresin such as gingerol are completely dependent on cyclodextrin, the actual pre-coating amount will not be very large, and the gingerol content in the final product will not be high. In order to increase the content of gingerol, the proportion of ginger oleoresin must be increased. In this way, during the cyclodextrin pre-coating process, there will still be a large amount of gingerol in the ginger oleoresin-cyclodextrin dispersion that cannot be coated by cyclodextrin, which will lead to a significant spicy taste of the product and cannot meet actual needs. In order to solve the above technical problems, the present invention is based on the pre-coating of cyclodextrin molecules, and further combines microcapsule embedding technology, using modified starch and the like for microcapsule emulsification and secondary embedding, thereby achieving the purpose of increasing the content of gingerol, effectively reducing the spicy taste, and improving water solubility and stability.

Additionally, gingerol in ginger oleoresin is easily converted into shogaol (which is much spicier than gingerol) in heated and low pH environments. Therefore, during the microcapsule emulsification and secondary embedding processes, the present invention effectively prevents the conversion of gingerol into shogaol by carefully controlling the pH of the liquid, thereby increasing the gingerol content and avoiding an increase in spiciness. The addition of flavonoids further enhances the stability of the product.

Compared with the prior art, the present invention offers the following advantages and effects: (1) The present invention employs a method for preparing water-soluble ginger oleoresin microcapsules with high gingerol content and low spiciness without the use of surfactants or chemical solvents. The absence of surfactants in the preparation process results in a product with high safety and low cost. Additionally, the absence of chemical solvents ensures a safer production process, facilitating the industrial production and scaling up of the product.

(2) The present invention combines cyclodextrin molecular pre-coating with microcapsule encapsulation technology, enhancing the encapsulation efficiency by controlling the pH of the liquid system and incorporating flavonoids. This approach maintains the stability of the gingerol component, reduces spiciness, and achieves the technical effects of high gingerol content, excellent water solubility, low spiciness, and a desirable thermal sensation.

(3) Following the homogenization of the ginger oleoresin emulsion, the present invention employs a constant temperature static fusion method to further enhance the effective coating of the colloid on the ginger oleoresin, thereby improving the product's encapsulation rate and ensuring stability during application.

(4) The water-soluble ginger oleoresin microcapsules prepared by the present invention exhibit a high gingerol content, with the gingerol content in the final product reaching up to 11.7%. These microcapsules demonstrate significantly improved water solubility, producing a clear and transparent aqueous solution with low spiciness. Additionally, the microcapsules show excellent stability in low pH environments, remaining clear without turbidity or precipitation in acidic solutions, thereby significantly improving pH tolerance and overall stability.

(5) The water-soluble ginger oleoresin microcapsules prepared by the present invention can meet the specific requirements of food products (such as beverages, oral liquids, candies, solid drinks, cold drinks, etc.), medicines, and cosmetics, and offer broad application prospects.

### DESCRIPTION OF THE DRAWING

FIG. 1 is a photo showing the dissolution effect of ginger oleoresin in water.

FIG. 2 shows the dissolution effect of water-soluble ginger oleoresin microcapsules obtained in Examples 1 to 3 in a neutral aqueous solution, with Examples 1 to 3 shown from left to right.

FIG. 3 shows the dissolution effect of water-soluble ginger oleoresin microcapsules obtained in Comparative Examples 1 to 5 in a neutral aqueous solution, with Comparative Examples 1 to 5 shown from left to right.

FIG. 4 shows the dissolution effect of water-soluble ginger oleoresin microcapsules obtained in Examples 1 to 3 in an acidic aqueous solution, with Examples 1 to 3 shown from left to right.

FIG. 5 shows the dissolution effect of water-soluble ginger oleoresin microcapsules obtained in Comparative Examples 1 to 5 in an acidic aqueous solution, with Comparative Examples 1 to 5 shown from left to right.

FIG. 6 shows the particle size distribution of water-soluble ginger oleoresin microcapsules obtained in Examples 1 to 3.

FIG. 7 shows the particle size distribution of water-soluble ginger oleoresin microcapsules obtained in Comparative Examples 1 to 5.

### DETAILED DESCRIPTION OF EMBODIMENTS

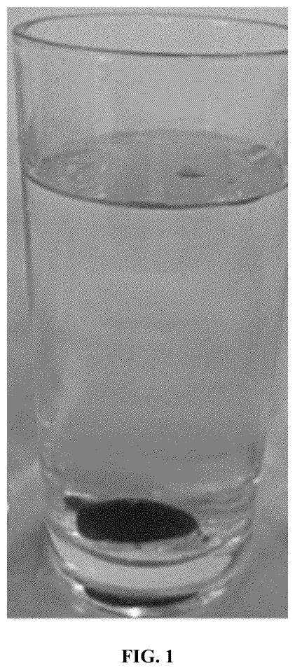

The present invention is further described in detail below in conjunction with the examples and drawings; however, the embodiments of the present invention are not limited to these. Unless otherwise specified, the technical methods used in the examples are conventional methods known to those skilled in the art. Unless otherwise specified, the reagents, methods, and equipment used in the present invention are conventional in the field. In this example, the ginger oleoresin used is commercially available, with a gingerol content of 41.6%. Its solubility in a neutral aqueous solution is shown in FIG. 1.

### Example 1

(1) Placing ginger oleoresin with a gingerol content of 41.6% in a 50° C. environment and keeping it still; weighing 28 g of α-cyclodextrin and 10 g of β-cyclodextrin, adding them to 52 g of deionized water, heating to 75° C. in a water bath, and keeping the solution completely dissolved; then adding 63 g of the above ginger oleoresin under high shear, fully emulsifying and dispersing (emulsification and dispersion speed of 3000 rpm, time of 16 minutes, emulsion temperature of 72-78° C.). Ginger oleoresin-cyclodextrin dispersion was obtained;
(2) Weighing 60 g of gum arabic, 6 g of gum dawa, 23 g of oligomaltose, and 39 g of maltodextrin. Adding these to 314 g of deionized water, stirring until evenly mixed, to obtain a wall material solution with a solid content of 28.9%. Heating the wall material solution to 70° C. in a water bath;
(3) Maintaining the temperature of the wall material solution from step (2) and adding the ginger oleoresin-cyclodextrin dispersion obtained in step (1) under high shear conditions. Fully emulsifying and dispersing the mixture at an emulsification speed of 4500 rpm for 20 minutes, with the emulsion temperature maintained at 69-75° C. Simultaneously, adding sodium carbonate and sodium hexametaphosphate to adjust the system's pH to 7.9. Then, adding EGCG (at 0.1% of the final product) and continuing emulsifying and dispersing until fully dissolved, forming a uniform ginger oleoresin emulsion. Finally, subjecting the ginger oleoresin emulsion to high-pressure homogenization (Homogenizing pressure at 75 MPa, homogenized twice) to obtain a stable ginger oleoresin homogenate;
(4) Allowing the ginger oleoresin homogenate obtained in step (3) to stand at a constant temperature of 45° C. for 5 hours, and then spray-drying, with the drying air inlet temperature maintained at 170-195° C. and the air outlet temperature maintained at 80-95° C., to obtain water-soluble ginger oleoresin microcapsules.

After testing, the water-soluble ginger oleoresin microcapsules demonstrated excellent water solubility, with the solution being clear and transparent. The gingerol content was 10.9 wt. %, and the encapsulation rate was 97.6%. Both the oral cavity and throat spiciness were low.

### Example 2

(1) Placing ginger oleoresin with a gingerol content of 41.6% in a 50° C. environment and keeping it still; weighing 20 g of β-cyclodextrin and 22 g of γ-cyclodextrin, adding them to 52 g of deionized water, heating to 75° C. in a water bath, and keeping the solution completely dissolved; then adding 68 g of the above ginger oleoresin under high shear, fully emulsifying and dispersing (emulsification and dispersion speed of 3500 rpm, time of 15 minutes, emulsion temperature of 72-78° C.). Ginger oleoresin-cyclodextrin dispersion was obtained;
(2) Weighing 70 g of sodium starch octenyl succinate, 20 g of pullulan, 15 g of maltitol, and 40 g of maltodextrin. Adding these to 315 g of deionized water, stirring until evenly mixed, to obtain a wall material solution with a solid content of 31.5%. Heating the wall material solution to 73° C. in a water bath;
(3) Maintaining the temperature of the wall material solution from step (2) and adding the ginger oleoresin-cyclodextrin dispersion obtained in step (1) under high shear conditions. Fully emulsifying and dispersing the mixture at an emulsification speed of 5000 rpm for 15 minutes, with the emulsion temperature maintained at 72-77° C. Simultaneously, adding potassium carbonate and sodium pyrophosphate to adjust the system's pH to 8.2. Then, adding myricetin (at 0.08% of the final product) and continuing emulsifying and dispersing until fully dissolved, forming a uniform ginger oleoresin emulsion. Finally, subjecting the ginger oleoresin emulsion to high-pressure homogenization (Homogenizing pressure at 80 MPa, homogenized twice) to obtain a stable ginger oleoresin homogenate;
(4) Allowing the ginger oleoresin homogenate obtained in step (3) to stand at a constant temperature of 45° C. for 5 hours, and then spray-drying, with the drying air inlet temperature maintained at 170-195° C. and the air outlet temperature maintained at 80-95° C., to obtain water-soluble ginger oleoresin microcapsules.

After testing, the water-soluble ginger oleoresin microcapsules demonstrated excellent water solubility, with the solution being clear and transparent. The gingerol content was 10.5 wt. %, and the encapsulation rate was 97.3%. Both the oral cavity and throat spiciness were low.

### Example 3

(1) Placing ginger oleoresin with a gingerol content of 41.6% in a 55° C. environment and keeping it still; weighing 22 g of α-cyclodextrin and 18 g of γ-cyclodextrin, adding them to 52 g of deionized water, heating to 75° C. in a water bath, and keeping the solution completely dissolved; then adding 78 g of the above ginger oleoresin under high shear, fully emulsifying and dispersing (emulsification and dispersion speed of 5500 rpm, time of 20 minutes, emulsion temperature of 72-78° C.). Ginger oleoresin-cyclodextrin dispersion was obtained;
(2) Weighing 50 g of sodium starch octenyl succinate, 10 g of gum arabic, 10 g of hydroxypropyl starch, 15 g of erythritol, 5 g of resistant dextrin, and 60 g of maltodextrin. Adding these to 315 g of deionized water, stirring until evenly mixed, to obtain a wall material solution with a solid content of 32.2%. Heating the wall material solution to 80° C. in a water bath;
(3) Maintaining the temperature of the wall material solution from step (2) and adding the ginger oleoresin-cyclodextrin dispersion obtained in step (1) under high shear conditions. Fully emulsifying and dispersing the mixture at an emulsification speed of 6000 rpm for 18 minutes, with the emulsion temperature maintained at 75-80° C. Simultaneously, adding potassium carbonate and sodium tripolyphosphate to adjust the system's pH to 8.3. Then, adding quercetin (at 0.12% of the final product) and continuing emulsifying and dispersing until fully dissolved, forming a uniform ginger oleoresin emulsion. Finally, subjecting the ginger oleoresin emulsion to high-pressure homogenization (Homogenizing pressure at 50 MPa, homogenized twice) to obtain a ginger oleoresin homogenate.
(4) Allowing the ginger oleoresin homogenate obtained in step (3) to stand at a constant temperature of 50° C. for 5 hours, and then spray-drying, with the drying air inlet temperature maintained at 170-195° C., and the air outlet temperature maintained at 80-95° C., to obtain water-soluble ginger oleoresin microcapsules.

After testing, the water-soluble ginger oleoresin microcapsules demonstrated excellent water solubility, with the solution being clear and transparent. The gingerol content was 11.7 wt. %, and the encapsulation rate was 98.3%. Both the oral cavity and throat spiciness were low.

### Comparative Example 1

### (Without Using Cyclodextrin for Pre-Coating, Directly Use Starch and Other Wall Materials for Microcapsule Embedding)

(1) Placing ginger oleoresin with a gingerol content of 41.6% in a 55° C. environment and keeping it still;
(2) The preparation of the wall material solution follows the same procedure as in Example 1;
(3) Maintaining the temperature of the wall material solution from step (2) and adding 63 g of the ginger oleoresin-cyclodextrin dispersion obtained in step (1) under high shear conditions. Fully emulsifying and dispersing the mixture at an emulsification speed of 4500 rpm for 20 minutes, with the emulsion temperature maintained at 69-75° C. Simultaneously, adding sodium carbonate and sodium hexametaphosphate to adjust the system's pH to 7.9. Then, adding EGCG (at 0.1% of the final product) and continuing emulsifying and dispersing until fully dissolved, forming a ginger oleoresin emulsion. Finally, subjecting the ginger oleoresin emulsion to high-pressure homogenization (Homogenizing pressure at 75 MPa, homogenized twice) to obtain a stable ginger oleoresin homogenate;
(4) Allowing the ginger oleoresin homogenate obtained in step (3) to stand at a constant temperature of 45° C. for 5 hours, and then spray-drying, with the drying air inlet temperature maintained at 170-195° C., and the air outlet temperature maintained at 80-95° C., to obtain water-soluble ginger oleoresin microcapsules.

After testing, the water-soluble ginger oleoresin microcapsules demonstrated general water solubility, a sightly turbid solution. The gingerol content was 9.1 wt. %, and the encapsulation rate was 75.7%. The oral cavity spiciness was low, but the throat spiciness was high.

### Comparative Example 2

### (Without Adding Myricetin)

(1) The preparation of the ginger oleoresin-cyclodextrin dispersion is the same as in Example 2;
(2) The preparation of the wall material solution is the same as in Example 2;
(3) Maintaining the temperature of the wall material solution from step (2) and adding the ginger oleoresin-cyclodextrin dispersion obtained in step (1) under high shear conditions. Fully emulsifying and dispersing the mixture at an emulsification speed of 5000 rpm for 15 minutes, with the emulsion temperature maintained at 72-77° C. Simultaneously, adding potassium carbonate and sodium pyrophosphate to adjust the system's pH to 8.2, forming a ginger oleoresin emulsion. The ginger oleoresin emulsion was then subjected to high-pressure homogenization (Homogenizing pressure at 80 MPa, homogenized twice) to obtain a ginger oleoresin homogenate;
(4) Allowing the ginger oleoresin homogenate obtained in step (3) to stand at a constant temperature of 45° C. for 5 hours, and then spray-drying, with the drying air inlet temperature maintained at 170-195° C., and the air outlet temperature maintained at 80-95° C., to obtain water-soluble ginger oleoresin microcapsules.

After testing, the water-soluble ginger oleoresin microcapsules demonstrated excellent water solubility, with the solution being clear and transparent. The gingerol content was 8.7 wt. %, and the encapsulation rate was 81.6%. The oral cavity spiciness was low, but the throat spiciness was medium.

### Comparative Example 3

### (The pH of the Emulsion was not Adjusted to 8.2)

(1) The preparation of the ginger oleoresin-cyclodextrin dispersion is the same as in Example 2;
(2) The preparation of the wall material solution is the same as in Example 2;
(3) Maintaining the temperature of the wall material solution from step (2) and adding the ginger oleoresin-cyclodextrin dispersion obtained in step (1) under high shear conditions. Fully emulsifying and dispersing the mixture at an emulsification speed of 5000 rpm for 15 minutes, with the emulsion temperature maintained at 72-77° C. Then, adding myricetin (at 0.08% of the final product) and continuing emulsifying and dispersing until fully dissolved, forming a uniform ginger oleoresin emulsion. Finally, subjecting the ginger oleoresin emulsion to high-pressure homogenization (Homogenizing pressure at 80 MPa, homogenized twice) to obtain a stable ginger oleoresin homogenate;
(4) Allowing the ginger oleoresin homogenate obtained in step (3) to stand at a constant temperature of 45° C. for 5 hours, and then spray-drying, with the drying air inlet temperature maintained at 170-195° C., and the air outlet temperature maintained at 80-95° C., to obtain water-soluble ginger oleoresin microcapsules.

After testing, the water-soluble ginger oleoresin microcapsules demonstrated general water solubility, a turbid solution. The gingerol content was 8.8 wt. %, and the encapsulation rate was 79.2%. Both the oral cavity and throat spiciness were high.

### Comparative Example 4

### (Without Constant Temperature Static Fusion, Direct Drying was Performed)

(1) The preparation of the ginger oleoresin-cyclodextrin dispersion is the same as in Example 3;
(2) The preparation of the wall material solution is the same as in Example 3;
(3) The preparation of the ginger oleoresin homogenate is the same as in Example 3;
(4) Directly spray-drying the ginger oleoresin homogenate obtained in step (3)., with the drying air inlet temperature maintained at 170-195° C., and the air outlet temperature maintained at 80-95° C., to obtain water-soluble ginger oleoresin microcapsules.

After testing, the water-soluble ginger oleoresin microcapsules demonstrated excellent water solubility, with the solution being clear and transparent. The gingerol content was 9.8 wt. %, the embedding rate was 88.3%, and both the oral cavity and throat spiciness were medium.

### Comparative Example 5

(Encapsulation Using Cyclodextrin Alone, without Employing Microencapsulation technology)
(1) Placing ginger oleoresin with a gingerol content of 41.6% in a 50° C. environment and keeping it still; weighing 38 g of α-cyclodextrin and 26 g of β-cyclodextrin, adding them to 366 g of deionized water, heating to 75° C. in a water bath, and keeping the solution completely dissolved; then adding 63 g of the above ginger oleoresin under high shear, fully emulsifying and dispersing (emulsification and dispersion speed of 3000 rpm, time of 16 minutes, emulsion temperature of 72-78° C.). Ginger oleoresin-cyclodextrin dispersion was obtained;
(2) The ginger oleoresin-cyclodextrin dispersion is then subjected to high-pressure homogenization (Homogenizing pressure at 75 MPa, homogenized twice) to obtain a ginger oleoresin homogenate;
(3) The homogenized ginger oleoresin liquid obtained in step (2) was allowed to stand at a constant temperature of 45° C. for 5 hours and then spray-dried. (Note: After standing at a constant temperature for 5 hours, a distinct layer of ginger oleoresin was observed floating on the surface of the emulsion, with a small amount of sediment at the bottom.) The lower part of the emulsion was then filtered through a 100-mesh filter to remove the sediment, and the filtrate was spray-dried. The drying air inlet temperature maintained at 170-195° C., and the air outlet temperature maintained at 80-95° C., yielding water-soluble ginger oleoresin microcapsules.

After testing, the water-soluble ginger oleoresin microcapsules demonstrated general water solubility, a turbid solution. The gingerol content was 6.1 wt. %, and the encapsulation rate was 33.6%. Both the oral cavity and throat spiciness were high.

### Effect Example

### (1) Determination of Encapsulation Rate

(a) Determination of Gingerol Content in Microcapsules: Accurately weighing 5 g of the water-soluble ginger oleoresin microcapsule sample. Adding 15 mL of water to form a uniform system, then centrifuging at 4000 rpm for 30 minutes. Decanting the supernatant, tilting the centrifuge bucket to allow the surface moisture of the residue at the bottom to drain, and then adding 35 mL of anhydrous ethanol to dissolve the ginger oleoresin. Determining the gingerol content by spectrophotometry at 280 nm.

(b) Determination of Gingerol Content on Microcapsule Surface: Accuratel weighing 5 g of the water-soluble ginger oleoresin microcapsule sample. Adding 25 mL of anhydrous ethanol, stirring on a stirrer for 5 minutes, filtering the mixture with filter paper, and rinsing the microcapsules on the filter paper with anhydrous ethanol to wash out the surface gingerol, and diluting to 50 mL. Determining the gingerol content by spectrophotometry at 280 nm.

(c) Calculation of Encapsulation Rate: embedding rate (%)=(1-microcapsule surface gingerol content)×100/microcapsule gingerol content.

(2) Measurement of Spiciness: The water-soluble ginger oleoresin microcapsule samples prepared in Examples 1 to 3 and Comparative Examples 1 to 5 were sequentially numbered 1 to 8, and ginger oleoresin solutions of the same concentration (0.2 wt. %) were prepared using purified water. Eighteen participants were randomly selected to taste the 8 groups of aqueous solutions in sequence, evaluating the spiciness in the mouth and throat. The participants chose from three options: high, medium, and low. The spiciness level was determined by aggregating the evaluations of the 18 participants. Boiled water was used to rinse the mouth between tastings to prevent cross-sensory influence from the previous solution.

(3) Content of Gingerol in Microcapsules: Weighing an appropriate amount of the water-soluble ginger oleoresin microcapsule sample and ultrasonicating it with 15 mL of water for 10 minutes, ensuring complete dissolution. Transferring the solution to a 50 mL volumetric flask and adding methanol up to the mark, preparing the sample solution. Following the national standard “GB/T 22293-2008 Ginger and its oleoresin-Determination of main irritant components-HPLC method” for the preparation of the standard substance solution and the specific determination method.

(4) Determining Microcapsule Particle Size: Determining the particle size of microcapsules by following the “GB/T 19077-2016 Particle Size Distribution-Laser Diffraction Method”.

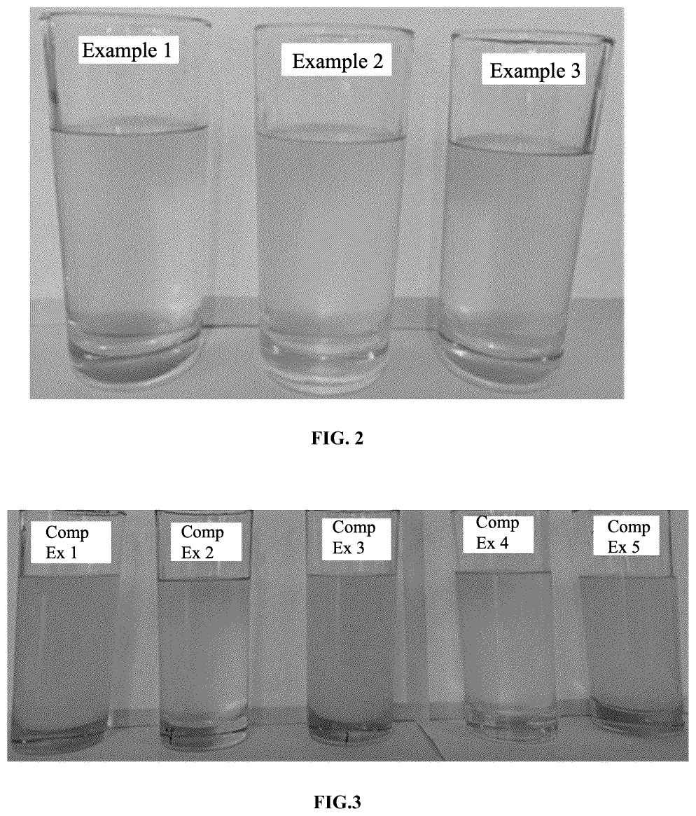

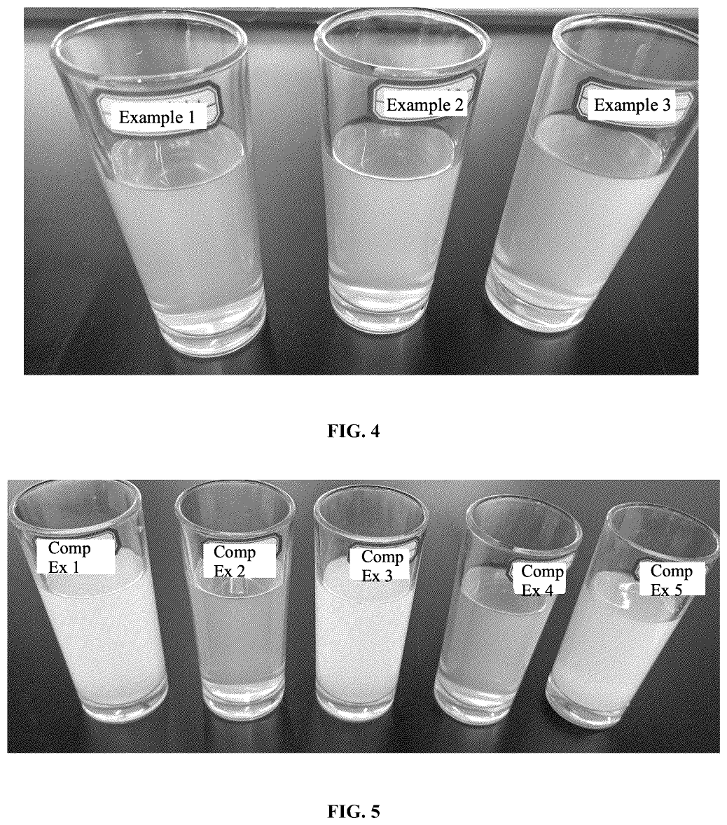

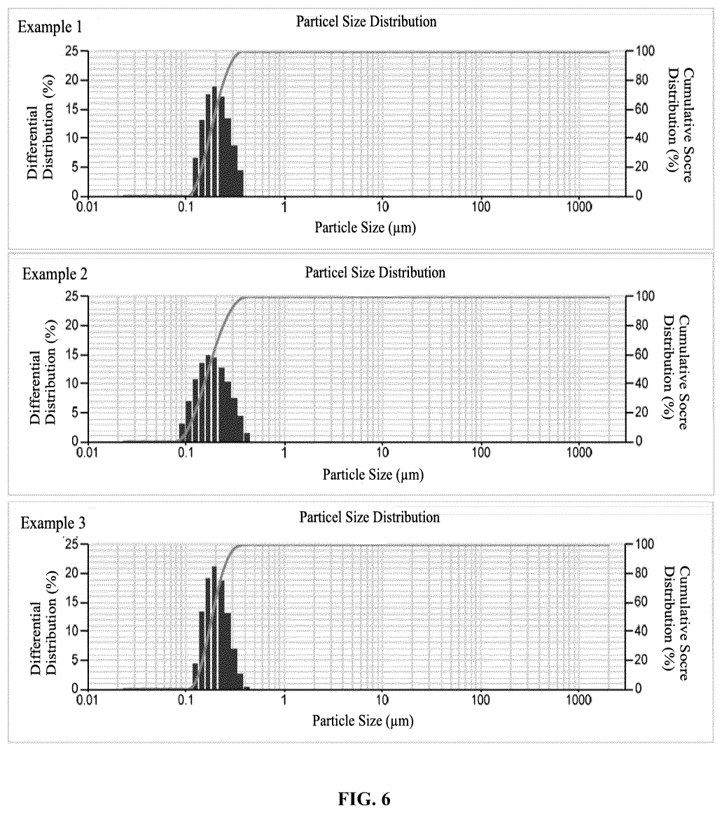

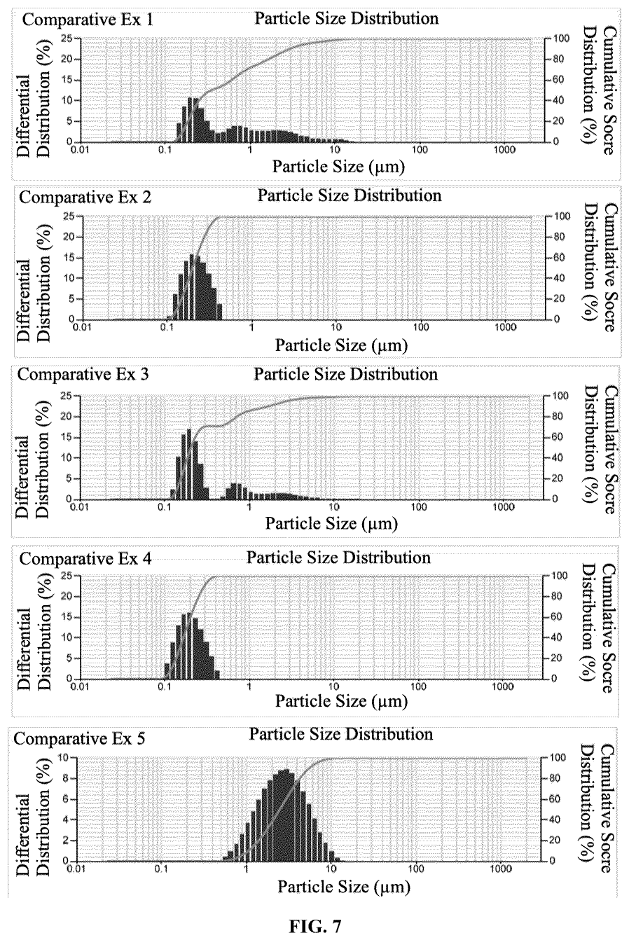

(5) Solubility Test and Stability Evaluation Method in Low pH Solution Environment: Accurately weighing 0.1500 g of the water-soluble ginger oleoresin microcapsule sample, adding a small amount of distilled water or acidic water (prepared by dissolving 0.5 g citric acid and 0.3 g sodium citrate in 500 g distilled water) and stirring. After dissolution, adding distilled water or acidic water to reach a total of 150 g. After stirring evenly, judging the solubility and stability based on the state of the solution (clear or turbid) after standing for 12 hours. The experimental results are shown in Table 1 and FIGS. 2, 3, 4, 5, 6, and 7.

The difference between Comparative Example 1 and Example 1 lies in the use of cyclodextrin for pre-coating in Example 1, while in Comparative Example 1, starch and other wall materials are directly used for microcapsule embedding without pre-coating. As shown in FIGS. 2 and 3, the ginger oleoresin microcapsules prepared in Example 1 exhibit high solubility in both water and acidic solutions, with clear and transparent solutions, and 90% of the particle sizes are below 0.5 μm. In contrast, the microcapsules in Comparative Example 1 are generally soluble in water and acidic solutions, the solutions are relatively turbid, especially in acidic environments. Additionally, Comparative Example 1 shows lower gingerol content and embedding rate, with relatively larger and unevenly distributed particle sizes. Although the spiciness in the mouth (oral cavity) is low, the spiciness in the throat after swallowing is relatively strong.

The difference between Comparative Example 2 and Example 2 is the absence of myricetin in Comparative Example 2. While its solubility in water and acidic solutions is relatively high and the particle size distribution is uniform, with 90% of the particles below 0.5 μm, the gingerol content and embedding rate are lower. Additionally, the spiciness in the throat after swallowing is more pronounced compared to Example 2.

The difference between Comparative Example 3 and Example 2 is that the pH of the system is not adjusted during the emulsification and dispersion of the ginger oleoresin-cyclodextrin dispersion and the wall material solution. As a result, the spiciness in the oral cavity and throat after swallowing is very pronounced, and the gingerol content and encapsulation rate are low. Additionally, the particle size is large and unevenly distributed.

The difference between Comparative Example 4 and Example 3 is that the homogenized ginger oleoresin liquid is not subjected to constant temperature static fusion. Although the water solubility and particle size of the microcapsules are not affected, the gingerol content and encapsulation rate are lower, and the spiciness in the mouth and throat is more pronounced compared to Example 3.

In Comparative Example 5, only a higher amount of cyclodextrin is used for coating, without employing microcapsule embedding technology. The experimental results indicate that using cyclodextrin alone for coating leads to issues such as low gingerol content, low encapsulation rate, strong spiciness, poor water solubility, and low stability in low pH solutions.

TABLE 1
The difference between the examples and the comparative examples

| | | | | | | Spiciness | |
|---|---|---|---|---|---|---|---|
| Test | | Gingerol | | | Embedding | Oral | |
| item | Main process parameters | content | | Solubility | rate | cavity | Throat |
| Ex. 1 | Adding the ginger oleoresin- | 10.9 | wt. % | Excellent water solubility, a | 97.6% | Low | Low |
| | cyclodextrin dispersion to the | | | clear and transparent solution | | | |
| | wall material solution, | | | without precipitation | | | |
| | maintaining the emulsion | | | | | | |
| | temperature at 69-75° C., | | | | | | |
| | emulsifying for 20 minutes, | | | | | | |
| | pH value of 7.9. Adding | | | | | | |
| | EGCG, homogenizing twice at | | | | | | |
| | 75 MPa, then keeping at a | | | | | | |
| | constant temperature of 45° C. | | | | | | |
| | and drying. | | | | | | |
| Ex. 2 | Adding the ginger oleoresin- | 10.5 | wt. % | Excellent water solubility, a | 97.3% | Low | Low |
| | cyclodextrin dispersion to the | | | clear and transparent solution | | | |
| | wall material solution, | | | without precipitation | | | |
| | maintaining the emulsion | | | | | | |
| | temperature at 72-77° C., | | | | | | |
| | emulsifying for 15 minutes, | | | | | | |
| | pH value of 8.2. Adding | | | | | | |
| | myricetin, homogenizing | | | | | | |
| | twice at 80 MPa, then keeping | | | | | | |
| | at a constant temperature of | | | | | | |
| | 45° C. and drying. | | | | | | |
| Ex. 3 | Adding the ginger oleoresin- | 11.7 | wt. % | Excellent water solubility, a | 98.3% | Low | Low |
| | cyclodextrin dispersion to the | | | clear and transparent solution | | | |
| | wall material solution, | | | without precipitation | | | |
| | maintaining the emulsification | | | | | | |
| | temperature at 75-80° C., | | | | | | |
| | emulsifying for 18 minutes, | | | | | | |
| | pH value of 8.3. Adding | | | | | | |
| | quercetin, homogenizing | | | | | | |
| | twice at 50 MPa, then keeping | | | | | | |
| | at a constant temperature of | | | | | | |
| | 50° C. and drying. | | | | | | |
| Comp. | Adding ginger oleoresin to the | 9.1 | wt. % | General water solubility; | 75.7% | Low | High |
| Ex. 1 | wall material solution, | | | slightly turbid solution | | | |
| | maintaining the emulsion | | | | | | |
| | temperature at 69-75° C., | | | | | | |
| | emulsifying for 20 minutes, | | | | | | |
| | pH value of 7.9, adding | | | | | | |
| | EGCG, homogenizing twice at | | | | | | |
| | 75 MPa, then keeping at a | | | | | | |
| | constant temperature of 45° C. | | | | | | |
| | and drying. | | | | | | |
| Comps. | Adding ginger oleoresin- | 8.7 | wt. % | Excellent water solubility, a | 81.6% | Low | Medium |
| Ex. 2 | cyclodextrin dispersion to the | | | clear and transparent solution | | | |
| | wall material solution, | | | | | | |
| | maintaining the emulsion | | | | | | |
| | temperature at 72-77° C., | | | | | | |
| | emulsifying for 15 minutes, | | | | | | |
| | pH value of 8.2, | | | | | | |
| | homogenizing twice at 80 | | | | | | |
| | MPa, then keeping at a | | | | | | |
| | constant temperature of 45° C. | | | | | | |
| | before drying. | | | | | | |
| Comp. | Adding ginger oleoresin- | 8.8 | wt. % | General water solubility; a | 79.2% | High | High |
| Ex. 3 | cyclodextrin dispersion to the | | | turbid solution | | | |
| | wall material solution, | | | | | | |
| | emulsifying at 72° C. to 77° C. | | | | | | |
| | for 15 minutes, adding | | | | | | |
| | myricetin, homogenizing | | | | | | |
| | twice at 80 MPa, then keeping | | | | | | |
| | at a constant temperature of | | | | | | |
| | 45° C. before drying. | | | | | | |
| Comp. | Adding ginger oleoresin- | 9.8 | wt. % | Excellent water solubility, a | 88.3% | Medium | Medium |
| Ex. 4 | cyclodextrin dispersion to the | | | clear and transparent solution | | | |
| | wall material solution, | | | | | | |
| | maintaining the emulsification | | | | | | |
| | temperature between 75° C. and | | | | | | |
| | 80° C., pH value of 8.3, | | | | | | |
| | emulsifying for 18 minutes, | | | | | | |
| | adding quercetin, | | | | | | |
| | homogenizing twice at 50 | | | | | | |
| | MPa, and then drying. | | | | | | |
| Comp. | Preparing ginger oleoresin- | 6.1 | wt. % | General water solubility; a | 33.6% | High | High |
| Ex. 5 | cyclodextrin dispersion, | | | turbid solution | | | |
| | homogenizing twice at 75 | | | | | | |
| | MPa, then keeping at a | | | | | | |
| | constant temperature of 45° C. | | | | | | |
| | before drying. | | | | | | |

The above embodiments represent preferred implementations of the present invention, but the implementation of the present invention is not limited to these embodiments. Any changes, modifications, substitutions, combinations, or simplifications that do not deviate from the essence and principles of the present invention are considered equivalent replacements and fall within the scope of protection of the present invention.

1. A method for preparing water-soluble ginger oleoresin microcapsules, wherein the method comprises the following steps:
(1) mixing cyclodextrin with water and heating until completely dissolved to obtain a cyclodextrin solution; then adding ginger oleoresin under high shear conditions to emulsify and disperse, resulting in a ginger oleoresin-cyclodextrin dispersion;
(2) mixing the wall material with water to prepare a solution, then heating it to obtain a wall material solution;
(3) adding the ginger oleoresin-cyclodextrin dispersion obtained in step (1) to the wall material solution obtained in step (2) under high shear conditions to emulsify and disperse, and adjusting the system pH to 6.5-10.0; then adding flavonoids to continue emulsifying and dispersing to obtain a ginger oleoresin emulsion; high-pressure homogenizing the ginger oleoresin emulsion to obtain a ginger oleoresin homogenate;
(4) allowing the ginger oleoresin homogenate obtained in step (3) to stand at a constant temperature, and finally drying to obtain a water-soluble ginger oleoresin microcapsule.

2. The method for preparing water-soluble ginger oleoresin microcapsules according to claim 1, wherein the cyclodextrin used in step (1) is at least one of α-cyclodextrin, β-cyclodextrin, or γ-cyclodextrin.

3. The method for preparing water-soluble ginger oleoresin microcapsules according to claim 1, wherein the mass ratio of ginger oleoresin to cyclodextrin in step (1) is (1-2): 1.

4. The method for preparing water-soluble ginger oleoresin microcapsules according to claim 1, wherein the concentration of the cyclodextrin solution in step (1) is 10-50 wt. %.

5. The method for preparing water-soluble ginger oleoresin microcapsules according to claim 1, wherein the wall material in step (2) is at least one of gum arabic, pullulan, modified starch, gum dawa, peach gum, fucoidan, or chitosan.

6. The method for preparing water-soluble ginger oleoresin microcapsules according to claim 1, wherein the wall material solution in step (2) also includes a filler, wherein the filler is at least one of maltodextrin, microcrystalline cellulose, glucose syrup, microporous starch, resistant dextrin, white sugar, lactitol, erythritol, maltitol, sorbitol, stachyose, beet water-soluble fiber, trehalose, oligomaltose, or lactose.

7. The method for preparing water-soluble ginger oleoresin microcapsules according to claim 1, wherein the mass ratio of the ginger oleoresin-cyclodextrin dispersion to the wall material solution in step (3) is 1:(2.5-3).

8. The method for preparing water-soluble ginger oleoresin microcapsules according to claim 1, wherein the constant temperature standing in step (4) is performed at a temperature of 30-90° C. for 1-12 hours.

9. A water-soluble ginger oleoresin microcapsule, wherein the microcapsule is obtained by the preparation method according to claim 1.

10. An application of the water-soluble ginger oleoresin microcapsules according to claim 9 is in food, medicine, or cosmetics.
